(19) United States
(12) Patent Application Publication
(10) Pub. No.: US 2024/0189208 A1
(43) Pub. Date: Jun. 13, 2024

(54) Biocompatible Product Having A Matrix Comprising a Polysaccharide Co-Crosslinked With Chitosan

(71) Applicants: NOXELIS, Annecy (FR); FALGAGEN, Allonzier La Caille (FR)

(72) Inventors: Alexandre ADAMCZEWSKI, Cernex (FR); Gautier LALEVEE, Saint Pierre En Faucigny (FR)

(21) Appl. No.: 18/552,341

(22) PCT Filed: Apr. 19, 2022
(86) PCT No.: PCT/IB2022/053637

§ 371 Date: Sep. 25, 2023

(30) Foreign Application Priority Data
Apr. 26, 2021 (FR) FR2104295

(51) Int. Cl. A61K 8/73; A61K 8/27; A61K 9/00; A61K 31/722; A61K 31/728; A61K 31/737; A61K 47/02; A61L 27/44; A61Q 19/00

(52) U.S. Cl. CPC A61K 8/736; A61K 8/27; A61K 8/73; A61K 8/735; A61K 9/0048; A61K 31/722; A61K 31/728; A61K 31/737; A61K 47/02; A61L 27/446; A61Q 19/00; A61L 2400/06; A61L 2430/06; A61L 2430/34

(57) ABSTRACT

The invention relates to a biocompatible product having a crosslinked matrix, in which a polysaccharide is co-crosslinked with chitosan, a chitosan derivative, or a salt of chitosan, said matrix further comprising a divalent zinc cation.

### TECHNICAL FIELD OF THE INVENTION

The present invention relates to the field of biocompatible products for cosmetic and/or medical, in particular surgical, purposes. It relates, in particular, to a biocompatible product with a matrix comprising a polysaccharide that is at least partly co-crosslinked with chitosan, preferably a chitosan derivative or a salt of chitosan.

For a number of years now, there has been growing interest in products for filling, replacing or increasing the volume of a biological tissue and/or replacing or supplementing a biological fluid, in both the cosmetics and medical fields.

Thus, for example, the use of products enabling the increase in volume of biological tissue is particularly interesting in the cosmetics field, to treat fine lines and wrinkles but also to redefine the contours of the face and body.

Products for volume enhancement, filling, replacement of a biological tissue, or replacement or supplementation of a biological fluid may also be useful in the medical field.

Products designed to supplement synovial fluid in patients suffering from joint degeneration are, for example, known in the prior art. Such products most frequently comprise hyaluronic acid.

So-called “viscoelastic” products used in ophthalmic surgery to replace or supplement aqueous humor (during cataract surgery or in the framework of refractive surgery) or vitreous humor (in the case of vitrectomy), as described in U.S. Pat. No. 4,716,154 are also known.

The rheological properties of the product are of particular importance.

A certain number of biocompatible products, in particular products comprising crosslinked hyaluronic acid or a salt of crosslinked hyaluronic acid, have made it possible to fill, replace or increase the volume of a biological tissue and/or replace or supplement a biological fluid. However, some of them cannot be used effectively in either the cosmetic or medical fields due to their undesirable side effects and/or their weak efficacy.

The efficacy of some of these products could be improved by giving them higher rheological properties, and by also limiting the risks of degradation of their rheological properties.

In particular, it is useful and important for many applications for the product to exhibit a high viscosity, especially at low shear rates.

Products that have a base of crosslinked hyaluronic acid or of a salt of crosslinked hyaluronic acid exhibit a relatively satisfactory viscosity after crosslinking.

The viscosity of these products, however, tends to decrease sharply after fulfillment of heat treatment. Yet, these products must be sterilized before use in humans or animals, and this is generally achieved by heat treatment (for example, in an autoclave).

U.S. Pat. No. 10,898,613 B2 describes a biocompatible injectable hydrogel composition of crosslinked hyaluronic acid containing from 0.05 mM to 4 mM of divalent zinc cation to confer improved stability (assessed by a viscosity parameter) after autoclave sterilization.

FR 3 098 260 A1 describes the co-crosslinking of carboxymethyl chitosan with hyaluronan, with the objective of combining the recognized moisturizing properties of hyaluronan with the protective properties against oxidative stress of chitosan.

### DESCRIPTION OF THE INVENTION

A task proposed by the present invention is to provide a biocompatible product allowing to better fill, replace, increase the volume of a biological tissue and/or replace or supplement a biological fluid.

At the same time, the invention aims to provide such a product exhibiting improved rheological properties, in particular in terms of viscosity after use of a heat treatment such as autoclave sterilization.

To achieve these and other aims, the invention proposes a biocompatible product according to claim 1.

A “biocompatible product” in the sense of the present invention refers to any product that is well tolerated by a living organism and that does not cause a rejection reaction, toxic reaction, lesion or harmful effect to the biological functions of the living organism.

A “chitosan derivative” means a substituted chitosan exhibiting substitution of the D-glucosamine and/or the N-acetyl-D-glucosamine units, and in which the substitution group is bonded in a covalent manner.

The applicant has found that a co-crosslinking of a polysaccharide (selected from the group comprising hyaluronic acid, a salt of hyaluronic acid, chondroitin sulfate, cellulose derivatives, and a mixture thereof) with chitosan, a chitosan derivative or a salt of chitosan, which leads to the establishment of covalent bonds in the matrix between the polysaccharide and the chitosan, chitosan derivative or salt of chitosan, enables a substantial increase in the viscosity of the product when compared with the crosslinked polysaccharide. The applicant has, however, also found that the viscosity of said co-crosslinked product remained highly subject to degradation following a heat treatment such as autoclaving.

It was in this context that the applicant was surprised to discover that the co-crosslinking of a polysaccharide (selected from the group comprising hyaluronic acid, a salt of hyaluronic acid, chondroitin sulfate, cellulose derivatives, and a mixture thereof) with chitosan, a chitosan derivative or a salt of chitosan, combined with the presence of a divalent zinc cation, not only significantly increases the viscosity of the product, but moreover effectively limits the decrease in viscosity after heat treatment such as autoclaving. This lesser decrease in viscosity moreover bodes well for improved durability and better suitability for filling, replacing, increasing the volume of a biological tissue and/or replacing, supplementing a biological fluid.

According to a first possibility, the divalent zinc cation may be introduced by the addition of a zinc saccharide to be crosslinked in the matrix. For the purposes of this invention, “crosslinked in the matrix” means bonded to at least one of the constituents of the matrix by at least two covalent bonds.

According to another possibility, the divalent zinc cation may be introduced by the addition of a zinc saccharide to be grafted in the matrix. For the purposes of this invention. “grafted in the matrix” means linked to at least one of the constituents of the matrix by a covalent bond.

According to yet another possibility, the divalent zinc cation may preferably be dispersed in the matrix. “Dispersed in the matrix” is understood to mean an absence of covalent bonding to the constituents of the matrix. A dispersion of the divalent zinc cation in the matrix has produced excellent results in terms of viscosity. The method of manufacturing is, moreover, simpler than in the case of addition for the purposes of crosslinking or grafting, since zinc (in the form of a salt of zinc or, in particular, in the form of a zinc saccharide) exhibits a relatively poor solubility in the pH range required for crosslinking or grafting.

Advantageously, for a good increase in viscosity, the concentration of divalent zinc cation in the matrix may be between 0.001% and 1% by weight relative to the total weight of the matrix, and preferably between 0.01% and 1% by weight relative to the total weight of the matrix.

Preferably, in order to maintain good transparency of the product for applications requiring it (particularly in the ophthalmological field or for injections close to the skin surface), the concentration of divalent zinc cation in the matrix may be less than or equal to 0.1% by weight relative to the total weight of the matrix.

Advantageously, the concentration of divalent zinc cation in the matrix may be between 0.002% and 0.06% by weight relative to the total weight of the matrix, and preferably between 0.01% and 0.6% by weight relative to the total weight of the matrix. With such a concentration, the applicant has noted with great surprise but also great interest that the viscosity of the product according to the invention after heat treatment (such as autoclaving) remains high, may be close to its viscosity before heat treatment, or may even be higher than its viscosity before heat treatment.

Preferably, the divalent zinc cation may be included in the matrix by the addition of a salt of zinc or zinc saccharide, preferably zinc gluconate, zinc chloride or zinc sulfate. Zinc gluconate promotes the obtainment of a product with good transparency, particularly for applications requiring it (notably in the ophthalmological field or for injections close to the skin surface). Zinc chloride and zinc sulfate have also been proven to be useful in rheumatology, in orthopedic surgery, or for the treatment of osteoarthritis or for the repair of cartilage defects.

Advantageously, it may be provided that:
the concentration of polysaccharide selected from the group comprising hyaluronic acid, a salt of hyaluronic acid, chondroitin sulfate, cellulose derivatives, and a mixture thereof, is between 0.01% and 3% by weight relative to the total weight of the matrix, and/or
the concentration of chitosan, chitosan derivative or salt of chitosan is between 0.01% and 3% by weight, based on the total weight of the matrix.

Preferably, the chitosan derivative may be a carboxyalkyl chitosan. Good results have, in particular, been obtained with carboxymethyl chitosan.

Carboxyalkyl chitosan refers to a chitosan exhibiting carboxyalkyl-substituted glucosamine units, N-acetyl glucosamine units and glucosamine units. This is referred to as chitosan derivative or substituted chitosan.

Advantageously, the polysaccharide may be a hyaluronic acid or a salt of hyaluronic acid, and may preferably exhibit a molecular weight of between 0.1 and 5 MDa.

According to another aspect of the present invention, a cosmetic or pharmaceutical composition is proposed comprising a biocompatible product as previously described. This cosmetic or pharmaceutical composition may be in the form of a viscoelastic solution or a hydrogel.

Advantageously, the cosmetic or pharmaceutical composition may comprise a physiologically acceptable buffer, preferably containing polyols (mannitol, sorbitol, glycerol) and/or phospholipids.

Preferably, said composition may be used in a cosmetic or therapeutic treatment method, comprising, in particular, topical application, implantation, instillation or subcutaneous, intradermal, mucosal, ocular, intraocular or intraarticular injection of said composition.

Advantageously, said composition may be used for the prevention and/or treatment of pathologies that may be improved or avoided by:
filling or replacing a biological tissue; and/or
increasing the volume of said biological tissue; and/or
supplementing or replacing a biological fluid.

Preferably, said composition may be used for a method of treating osteoarthritis, or for the repair of a cartilage defect, wherein the composition is for example formulated for administration by injection into synovial fluid or for implantation into cartilage after mixing with blood, or for a method of treating dry eye.

Advantageously, said composition may be used in a therapeutic, surgical or cosmetic treatment method, including, in particular, treatment in rheumatology, ophthalmology, gynecology, esthetic medicine, plastic surgery, internal surgery, orthopedic surgery, for the prevention of post-surgical tissue adhesions, and in dermatology.

More particularly preferred, the composition may be used in the therapeutic treatment of dry eye syndrome, of corneal injury or of ocular or joint inflammation.

Advantageously, said composition may be used in a therapeutic treatment during which said composition is applied by instillation to the ocular surface to prevent or combat corneal injury or dry eye syndrome, in particular with the objective of lubricating or regenerating the ocular surface.

According to another aspect of the present invention, an eye drop composition is proposed comprising a biocompatible product as previously described.

According to another aspect of the present invention, a method for manufacturing a biocompatible product as previously described or a composition as previously described is proposed, said method comprising the following steps:
a. dissolution in the same aqueous solution of
   i. a polysaccharide selected from the group comprising hyaluronic acid, a salt of hyaluronic acid, chondroitin sulfate, cellulose derivatives, and a mixture thereof, preferably hyaluronic acid or a salt of hyaluronic acid,
   ii. chitosan, preferably a chitosan derivative, or a salt of chitosan,
b. addition of a crosslinking agent to said aqueous solution and the provocation of a crosslinking of the mixture to form a matrix,
c. addition of a divalent zinc cation before and/or after step b.

It should be noted that the invention is not limited to a particular covalent crosslinking method, but a method using a chemical molecule as crosslinking agent, such as, in particular, 1,4-butanediol diglycidyl ether (BDDE) or poly(ethylene glycol) diglycidyl ether (PEGDE) may be preferred.

When the divalent zinc cation is added prior to step b, this addition may be in the form of a zinc saccharide, in view of a crosslinking and/or a grafting in the matrix, or may be in the form of a salt of zinc to be dispersed in the matrix.

When the divalent zinc cation is added after step b, this addition is done in the form of a salt of zinc or zinc saccharide which is dispersed in the matrix.

The two preceding possibilities for the addition of divalent zinc cation may be combined.

Advantageously, the pH of the biocompatible product or of the composition may be adjusted to between 6 and 8, preferably 7.

Preferably, the biocompatible product or the composition may be subjected to a sterilizing heat treatment, preferably a heat treatment at a temperature of between 120° C. and 140° C. for a time of between 1 and 20 minutes.

Further objectives, features and advantages of the invention will become clear to those skilled in the art on reading the explanatory description, which refers to examples and embodiments which are given by way of illustration only and in no way limit the scope of the claims.

The examples and embodiments form an integral part of the present invention, and any feature which appears new in relation to any prior art from the description taken as a whole, including the examples and embodiments, forms an integral part of the invention in its function and on its whole.

Thus, each example or embodiment is broad in scope.

In the examples or in the embodiments, all percentages are, moreover, given in mass unless otherwise indicated, temperature is expressed in degrees Celsius unless otherwise indicated, and pressure is atmospheric pressure unless otherwise indicated.

### BRIEF DESCRIPTION OF THE DRAWINGS

Further objects, features and advantages of the present invention will become apparent from the following description of particular embodiments, made in connection with the accompanying figures, wherein:

FIG. 1 is a histogram of the viscosities of different formulations, measured before and after heat treatment; and

FIG. 2 is a graph illustrating the evolution of the viscosities of different formulations and products on the market as a function of shear rate.

### DESCRIPTION OF THE PREFERRED EMBODIMENTS

### Materials:

sodium hyaluronate (NaHa) derived from biofermentation,
carboxymethyl chitosan (CMCS) with a degree of acetylation of less than 10% (expressed as the number of moles of N-acetyl-glucosamine units relative to the number of moles of total units) and a degree of substitution of 80% (expressed as the number of moles of the substituent relative to the number of moles of total units).
1,4-butanediol diglycidyl ether (BDDE),
poly(ethylene glycol) diglycidyl ether (PEGDE),
zinc gluconate (ZnGluc),
zinc chloride (ZnCl2),
zinc sulfate (ZnSO₄),
sodium chloride (NaCl),
Physiological buffer,
hydrochloric acid (HCl),
sodium hydroxide (NaOH),
dialysis membranes.

### Preparation of a Physiological Buffer Solution

A physiological buffer solution is prepared in deionized water to achieve a pH of between 6 and 8 and an osmolarity of between 250 and 350 mOsm/L.

Synthesis of a Crosslinked Phase Comprising a Polysaccharide (which is to Say, Hyaluronic Acid or a Salt of Hyaluronic Acid)

To simplify the understanding of the reader, hyaluronic acid or a salt of hyaluronic acid is referred to as “HA.”

The crosslinking of HA is achieved by dissolution of the HA (for example, sodium hyaluronate (NaHa)) in a 0.25 mol/L aqueous soda solution until it is completely solubilized. A crosslinking agent (for example, BDDE or PEGDE) is then added and stirred to homogenize. The mixture is hermetically sealed and placed in a water bath at 50° C. for 3 hours to induce a crosslinking reaction of the HA. Covalent bonds are thus created between the crosslinking agent and the HA. Bridges may thus be formed between two chains of HA. The gel is then homogenized and neutralized in a vessel with the addition of an aqueous hydrochloric acid solution to form a homogeneous, transparent phase. Dialysis is then performed to eliminate the residual crosslinking agent and stabilize the pH and the osmolarity.

Synthesis of a Crosslinked Phase Comprising a Polysaccharide (which is to Say, Hyaluronic Acid or a Salt of Hyaluronic Acid) of Chitosan or a Salt of Chitosan

To simplify the understanding of the reader, hyaluronic acid or a salt of hyaluronic acid is referred to as “HA,” whereas chitosan, a chitosan derivative or a salt of chitosan is referred to as “CHITO.”

The crosslinking of HA with CHITO is achieved by dissolution of the HA (for example, sodium hyaluronate (NaHa)) and a “CHITO” (for example, carboxyalkyl chitosan) in a 0.25 mol/L aqueous soda solution until completely solubilized. A crosslinking agent (for example, BDDE or PEGDE) is then added and stirred to homogenize. The mixture is hermetically sealed and placed in a water bath at 50° C. for 3 hours to induce a simultaneous crosslinking reaction (or co-crosslinking) of the HA and of the CHITO. “Simultaneous crosslinking” or “co-crosslinking” mean that covalent bonds may be created between the crosslinking agent and the HA, however likewise between the crosslinking agent and the CHITO. Bridges may thus be formed between two chains of HA, between two chains of CHITO, or between one chain of HA and one chain of CHITO. The gel is then homogenized and neutralized in a vessel with the addition of an aqueous hydrochloric acid solution to form a homogeneous, transparent phase. Dialysis is then performed to eliminate the residual crosslinking agent and stabilize the pH and the osmolarity.

Synthesis of a Linear Phase (Non-Crosslinked) of a Polysaccharide (which is to Say Hyaluronic Add or Salt of Hyaluronic Acid) with a Divalent Zinc Cation (and, where Appropriate, Chitosan, a Chitosan Derivative or a Salt of Chitosan)

To simplify the understanding of the reader, hyaluronic acid or a salt of hyaluronic acid is referred to as “HA,” whereas chitosan, a chitosan derivative or a salt of chitosan is referred to as “CHITO.”

HA, taken alone or with a CHITO, is dissolved in a physiological buffer solution until completely solubilized to form a transparent viscoelastic solution. A divalent zinc cation may be added by incorporation (by dispersion) in the solution of a salt of zinc or of a zinc saccharide (in particular zinc gluconate, zinc chloride or zinc sulfate). A mixture enables dissolution and homogenization of the salt of zinc in the linear phase.

### Heat Treatment

Each formulation undergoes a heat treatment corresponding to a sterilization cycle. This heat treatment is carried out in an HMT 260 MB laboratory autoclave from HMC EUROPE. The sterilization plateau, which lasts 1 minute, is set to a temperature of 131° C. The total duration of the heat treatment, including temperature build-up, plateau and cooling, is approximately 22 to 23 minutes.

### Method for Measurement of Viscosity

The rheological characterizations are carried out using a Discovery “DHR2” hybrid rheometer from TA Instruments, equipped with a cone-plate geometry of 40 mm in diameter and having an angle of 2 degrees, as well as a 55 micron truncation. The rheometer is also equipped with a Peltier plate to control the temperature of the sample during measurement.

During a measurement, the sample is placed on the bottom plate and the geometry is lowered until the trim gap (105 μm) is reached. Excess product is removed with a spatula. The geometry is then lowered to 55 μm of the lower plate.

The method used consists of a flow ramp at 35° C. applying a shear rate varying between 1 s⁻¹ and 1000 s⁻¹, with 5 points per decade.

Viscosities for comparing the different formulations are then taken from the viscosity value corresponding to a shear rate of 8.6 s⁻¹.

Test No. 1: Formulation of a Biocompatible Product with Crosslinked HA Matrix without Divalent Zinc Cation Present in the Matrix

A formulation of HA comprising a crosslinked HA phase is prepared. The final concentration of HA is 0.9% by weight relative to the total weight of the matrix.

The crosslinked phase of HA is prepared as previously described.

Firstly, 1.12 g of sodium hyaluronate (NaHa) is weighed and dissolved in 8.7 g of 0.25 mol/L aqueous soda solution. After complete solubilization, the crosslinking agent is added dropwise in the solution. Mixing is performed to homogenize the reaction medium and enable homogeneous crosslinking. The vessel containing the mixture is then hermetically sealed and placed in a water bath at 50° C. for 3 hours. The gel is then stored until the following day at between 2° C. and 8° C.

The following day, 9.58 g of gel is collected and placed in a homogenizer, to which 87.4 g of hydrochloric acid (HCl) concentrated to 24 mmol/L was added. The medium is homogenized by means of a shearing mixture, which leads to a swelling and rapid neutralization of the pH. The medium that is homogenized and neutralized in this manner is collected and is then transferred to dialysis membranes that are closed using tongs, and said membranes are then placed in a buffer solution for a minimum of 24 hours.

The biocompatible product with crosslinked matrix thus obtained has a density of approximately 1, and is then transferred into 1 mL glass syringes.

The glass syringes are then subjected to a heat treatment as previously described, by autoclaving with a sterilization plateau of 1 minute at 131° C. The total duration of the heat treatment, including temperature build-up, plateau and cooling, is approximately 22 to 23 minutes.

The viscosity of the obtained biocompatible product with crosslinked matrix (formulation 1) is measured as described above.

Test No. 2: Formulation of a Biocompatible Product with Crosslinked HA Matrix with One Divalent Zinc Cation Present in the Matrix

An HA formulation is prepared comprising 90% crosslinked phase of HA and 10% non-crosslinked (linear) phase with HA base. The HA concentration is 0.9% by weight based on the total weight of the matrix. A divalent zinc cation is added by dispersion in the matrix.

The crosslinked phase of HA is prepared as previously described in connection with Test No. 1.

In parallel, a linear phase is prepared as previously described.

0.21 g of sodium hyaluronate (NaHa) is weighed, to which 20.0 g of buffer solution is added, and a mixing is performed.

The crosslinked phase and the linear (non-crosslinked) phase are then mixed in a ratio of 9:1. A divalent zinc cation is added to the matrix by incorporating zinc gluconate into the solution (by means of a dispersion). A mixture enables dissolution and homogenization of the salt of zinc in the linear phase.

Several concentrations of divalent zinc cation are applied, namely: 0.0022% (formulation 2a), 0.0072% (formulation 2b) and 0.0144% (formulation 2c) by weight relative to the total weight of the matrix.

The biocompatible product with crosslinked matrix obtained has a density of approx. 1, and is then transferred into 1 mL glass syringes.

The glass syringes are then subjected to a heat treatment as previously described, by autoclaving with a sterilization plateau for 1 minute at 131° C. The total duration of the heat treatment, including temperature build-up, plateau and cooling, is approximately 22 to 23 minutes.

The viscosity of the obtained biocompatible product with crosslinked matrix (formulations 2a, 2b and 2c) is measured as previously described.

Test No. 3: Formulation of a Biocompatible Product with a Co-Crosslinked HA and CHITO Matrix, with No Divalent Zinc Cation Present in the Matrix

A formulation of HA and CHITO comprising 90% co-crosslinked HA-CHITO phase is prepared. The HA and CHITO concentrations are 0.9% and 0.1% by weight respectively, based on the total weight of the matrix.

The HA-CHITO crosslinked phase is prepared as follows.

Firstly, 2.00 g of sodium hyaluronate (NaHa) is weighed. Next, 0.215 g of carboxymethyl chitosan is weighed and added to the sodium hyaluronate. The mixture of carboxymethyl chitosan and sodium hyaluronate is dissolved in 20 g of 0.25 mol/L aqueous soda solution. After complete solubilization, the crosslinking agent is added dropwise in the solution. Mixing is performed to homogenize the reaction medium and enable homogeneous co-crosslinking (or simultaneous crosslinking). The vessel containing the mixture is then hermetically sealed and placed in a water bath at 50° C. for 3 hours. The gel is then stored until the following day at between 2° C. and 8° C.

The following day, 22.02 g of gel is collected and placed in a homogenizer, to which 175.4 g of hydrochloric acid (HCl) concentrated to 28 mmol/L in aqueous solution is added. The medium is homogenized by means of a shearing mixture, which leads to a swelling and rapid neutralization of the pH. The medium that is homogenized and neutralized in this manner is collected and is then transferred to dialysis membranes that are closed using tongs, and said membranes are then placed in a buffer solution for a minimum of 24 hours.

In parallel, a linear phase is prepared as previously described, with HA and with CHITO.

0.20 g sodium hyaluronate (NaHa) and 22 mg carboxymethyl chitosan are mixed, to which 20.0 g buffer solution is added. A mixture is obtained.

The crosslinked phase and the linear (non-crosslinked) phase are then mixed in a ratio of 9:1.

The obtained biocompatible product with crosslinked matrix has a density of approx. 1, and is then transferred into 1 mL glass syringes.

The glass syringes are then subjected to a heat treatment as described above, by autoclaving with a sterilization plateau for 1 minute at 131° C. The total duration of the heat treatment, including temperature build-up, plateau and cooling, is approximately 22 to 23 minutes.

The viscosity of the obtained biocompatible product with crosslinked matrix (formulation 3) is measured as described above.

Test No. 4: Formulation of a Biocompatible Product with a Co-Crosslinked HA and CHITO Matrix, with a Divalent Zinc Cation Present in the Matrix by Addition of Zinc Gluconate

A formulation of HA and CHITO is prepared, comprising 90% co-crosslinked HA-CHITO phase and 10% non-crosslinked (linear) HA-CHITO-based phase. The HA and CHITO concentrations are 0.9% and 0.1% by weight respectively, based on the total weight of the matrix.

The HA-CHITO crosslinked phase is prepared as previously described in connection with Test No. 3.

In parallel, a linear phase with HA and with CHITO is prepared as previously described in connection with Test No. 3, except that after complete dissolution of the sodium hyaluronate (NaHa) and of the carboxymethyl chitosan, a divalent zinc cation is added to the matrix by incorporation into the zinc gluconate solution (by means of a dispersion). A mixing enables a dissolution and homogenization of said salt of zinc in the linear phase.

Several concentrations of divalent zinc cation are applied, namely: 0.0072% (formulation 4a) and 0.0144% (formulation 4b) by weight relative to the total weight of the matrix.

The crosslinked phase and the linear (non-crosslinked) phase are then mixed in a ratio of 9:1.

The biocompatible product with crosslinked matrix thus obtained has a density of around 1, and is then transferred into 1 mL glass syringes.

The glass syringes are then subjected to a heat treatment as described above, by autoclaving with a sterilization plateau for 1 minute at 131° C. The total duration of the heat treatment, including temperature build-up, plateau and cooling, is approximately 22 to 23 minutes.

The viscosity of the obtained biocompatible product with crosslinked matrix (formulations 4a and 4b) is measured as previously described.

Test No. 5: Formulation of a Biocompatible Product with a Co-Crosslinked HA and CHITO Matrix, with a Divalent Zinc Cation Present in the Matrix by Addition of Zinc Chloride

A formulation of HA and CHITO is prepared, comprising 90% co-crosslinked HA-CHITO phase and 10% non-crosslinked (linear) phase with HA-CHITO base. The HA and CHITO concentrations are 0.9% and 0.1% by weight respectively, based on the total weight of the matrix.

The HA-CHITO crosslinked phase is prepared as previously described in connection with Test No. 4.

In parallel, a linear phase with HA and CHITO is prepared as previously described in connection with Test No. 4, except that after complete dissolution of sodium hyaluronate (NaHa) and carboxymethyl chitosan, a divalent zinc cation is added to the matrix by incorporation into the solution of zinc chloride (ZnCl2) (by means of a dispersion). A mixing enables a dissolution and homogenization of said salt of zinc in the linear phase.

Several concentrations of divalent zinc cation are applied, namely: 0.0072% (formulation 5a), 0.0144% (formulation 5b) and 0.048% (formulation 5c) by weight relative to the total weight of the matrix.

The crosslinked phase and the linear (non-crosslinked) phase are then mixed in a ratio of 9:1.

The biocompatible product with crosslinked matrix thus obtained has a density of approximately 1, and is then transferred into 1 mL glass syringes.

The glass syringes are then subjected to a heat treatment as described above, by autoclaving with a sterilization plateau for 1 minute at 131° C. The total duration of the heat treatment, including temperature build-up, plateau and cooling, is approximately 22 to 23 minutes.

The viscosity of the obtained biocompatible product with crosslinked matrix (formulations 5a, 5b and 5c) is measured as described above.

Test No. 6: Formulation of a Biocompatible Product with a Co-Crosslinked HA and CHITO Matrix, with a Divalent Zinc Cation Present in the Matrix by Addition of Zinc Sulfate

A formulation of HA and CHITO is prepared, comprising 90% co-crosslinked HA-CHITO phase and 10% non-crosslinked (linear) phase with HA-CHITO base. The concentrations of HA and CHITO are 0.9% and 0.1% by weight respectively, based on the total weight of the matrix.

The crosslinked phase of HA-CHITO is prepared as previously described in connection with Test No. 4.

In parallel, a linear phase with HA and with CHITO is prepared as previously described in connection with Test No. 4, except that after complete dissolution of sodium hyaluronate (NaHa) and carboxymethyl chitosan, a divalent zinc cation is added to the matrix by incorporation into the solution of zinc sulfate (ZnSO₄) (by means of a dispersion). A mixing enables a dissolution and homogenization of said salt of zinc in the linear phase.

Several concentrations of divalent zinc cation are applied, namely: 0.0072% (formulation 8a), 0.0144% (formulation 6b) and 0.0360% (formulation 6c) by weight relative to the total weight of the matrix.

The crosslinked phase and the linear (non-crosslinked) phase are then mixed in a ratio of 9:1.

The biocompatible product with crosslinked matrix thus obtained has a density of around 1, and is then transferred into 1 mL glass syringes.

The glass syringes are then subjected to a heat treatment as described above, by autoclaving with a sterilization plateau for 1 minute at 131° C. The total duration of the heat treatment, including temperature build-up, plateau and cooling, is approximately 22 to 23 minutes.

The viscosity of the obtained biocompatible product with crosslinked matrix (formulations 6a, 6b and 6c) is measured as described above.

### Measurements of Viscosity Before and After Heat Treatment

The viscosities of formulations 1, 2a through 2c, 3, 4a, 4b, 5a through 5c and 6a through 6c were measured, as described above, using a Discovery “DHR2” hybrid rheometer from TA Instruments, before and after application of the heat treatment described above (sterilization with a plateau lasting 1 minute at a temperature of 131° C., total heat treatment time including temperature rise, plateau and cooling is approximately 22 to 23 minutes).

TABLE 1
Results in terms of viscosity before and after heat treatment

| | | | Viscosity | Viscosity | |
|---|---|---|---|---|---|
| | | | BEFORE | AFTER | |
| | | | the heat | the heat | |
| Formu- | | [Zn²⁺] | treatment | treatment | Vari- |
| lations | Type of matrix | (source) | (cP) | (cP) | ations: |
| 1 | Crosslinked | No | 677 | 271 | −59.97% |
| | HA | | | | |
| 2a | Crosslinked | 0.0022% | 918 | 495 | −46.08% |
| | HA | (ZnGluc) | | | |
| 2b | Crosslinked | 0.0072% | 932 | 496 | −46.78% |
| | HA | (ZnGluc) | | | |
| 2c | Crosslinked | 0.0144% | 978 | 571 | −41.62% |
| | HA | (ZnGluc) | | | |
| 3 | Co-crosslinked | No | 4560 | 912 | −80.00% |
| | HA + CHITO | | | | |
| 4a | Co-crosslinked | 0.0072% | 5218 | 2028 | −61.13% |
| | HA + CHITO | (ZnGluc) | | | |
| 4b | Co-crosslinked | 0.0144% | 4808 | 4907 | 2.06% |
| | HA + CHITO | (ZnGluc) | | | |
| 5a | Co-crosslinked | 0.0072% | 5297 | 4776 | −9.84% |
| | HA + CHITO | (ZnCl₂) | | | |
| 5b | Co-crosslinked | 0.0144% | 5018 | 4790 | −4.54% |
| | HA + CHITO | (ZnCl₂) | | | |
| 5c | Co-crosslinked | 0.048% | 2931 | 3926 | 33.95% |
| | HA + CHITO | (ZnCl₂) | | | |
| 6a | Co-crosslinked | 0.0072% | 5482 | 3084 | −43.74% |
| | HA + CHITO | (ZnSO₄) | | | |
| 6b | Co-crosslinked | 0.0144% | 4749 | 4903 | 3.24% |
| | HA + CHITO | (ZnSO₄) | | | |
| 6c | Co-crosslinked | 0.0360% | 3050 | 3922 | 28.59% |
| | HA + CHITO | (ZnSO₄) | | | |

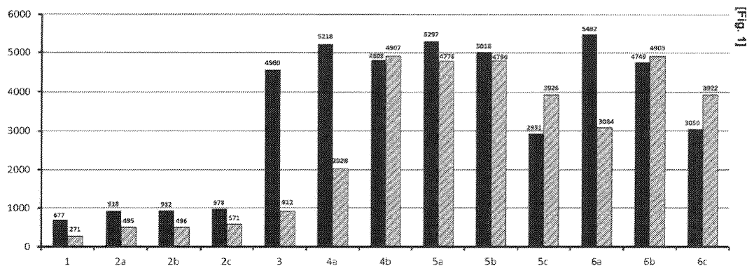

These results are shown graphically in FIG. 1 using a histogram. The abscissa shows the formulations tested, whereas the ordinate shows the viscosities measured (in cP). Viscosities before heat treatment are shown as solid bars, whereas viscosities after heat treatment are shown as hatched bars.

A first observation, based on formulations 1 and 2a to 2c, is that if the addition of a divalent zinc cation significantly increases the viscosity of a crosslinked matrix of HA, there is still a significant loss of viscosity after heat treatment. This loss seems almost constant (substantially equal to 400 cP in this example) and independent of the divalent zinc cation concentration.

A second observation, based on formulations 1 and 3, is that co-crosslinking of HA and of CHITO very significantly increases matrix viscosity before and after heat treatment. However, a significant drop (−80%) in viscosity persists after heat treatment.

A third observation, based on formulations 4a, 4b, 5a to 5c and 6a to 6c, is that the addition of a divalent zinc cation in a co-crosslinked matrix of HA and of CHITO at a relatively low concentration of between 0.002% and 0, 06% by weight, based on the total weight of the matrix, enables the matrix to retain a higher viscosity (at least twice as high) after heat treatment than in formulation 3 (co-crosslinked matrix of HA and of CHITO without divalent zinc cation).

The addition of a divalent zinc cation to a co-crosslinked matrix of HA and CHITO in a concentration of between 0.001% and 1% thus makes it possible to simultaneously obtain a much higher viscosity of the matrix and a much reduced loss of viscosity after heat treatment.

A fourth observation, based more specifically on formulations 4b, 5a to 5c, 6b and 6c, is that the addition of a divalent zinc cation to a co-crosslinked matrix of HA and CHITO at a concentration of between approx. 0.005% and approx. 0.06% results in a viscosity after heat treatment that is almost identical to the viscosity before heat treatment (formulations 5a, 5b), or even higher than the viscosity before heat treatment (formulations 4b, 5c, 6b and 6c).

This last observation is very surprising: conventionally, heat treatment of a crosslinked matrix tends to degrade the cohesion of said matrix and therefore to lower its viscosity. This unexpected phenomenon, which may even lead to an “inversion of viscosity” (meaning that the viscosity of the matrix is higher after heat treatment than before), is therefore particularly advantageous for all applications requiring sterilization of a composition comprising the biocompatible product, in particular for use in a cosmetic or therapeutic treatment method, including topical application, implantation, instillation or subcutaneous, intradermal, mucosal, ocular, intraocular or intra-articular injection of said composition.

The present invention, which enables a high viscosity after heat treatment, or even higher than the viscosity before heat treatment, reveals itself to be very valuable for the prevention and/or treatment of pathologies that may be improved or avoided by:
filling or replacing biological tissue; and/or
Increasing the volume of said biological tissue; and/or
supplementing or replacing a biological fluid.

The present invention, which enables a high viscosity to be obtained after heat treatment, or even higher than the viscosity prior to heat treatment, is also highly appreciable for a method of treating osteoarthritis, or repairing a cartilage defect, the composition being formulated, for example, for administration by injection into the synovial fluid or for implantation into the cartilage after mixing with blood, or for a method of treating dry eyes.

The present invention, which enables a high viscosity to be obtained after heat treatment, or even higher than the viscosity before heat treatment, is also highly appreciable for use in a therapeutic, surgical or cosmetic treatment method, including in particular treatment in rheumatology, ophthalmology, gynecology, esthetic medicine, plastic surgery, internal surgery, orthopedic surgery, for the prevention of post-surgical tissue adhesions, and in dermatology.

The present invention, which enables a high viscosity to be obtained after heat treatment, or even higher than the viscosity prior to heat treatment, is also proving highly appreciable for use in the therapeutic treatment of dry eye syndrome, corneal injury or ocular or joint inflammation.

The present invention, which enables a high viscosity to be obtained after heat treatment, or even higher than the viscosity prior to heat treatment, is also highly appreciable for use in therapeutic treatment during which said composition is applied by instillation to the ocular surface to prevent or combat corneal injury or dry eye syndrome, in particular with the objective of lubricating or regenerating the ocular surface.

An eye drop composition may, in particular, be produced comprising a biocompatible product with a co-crosslinked matrix of HA and CHITO as described above.

### Biocompatibility Testing by Intracutaneous Injection

Biocompatibility testing (adapting ISO standard 10993—part 10) was carried out by intracutaneous injections of 0.2 mL doses on the backs of rabbits at separate sites. The sites were observed immediately after injection. Observations were made in terms of erythema and edema after 24 h (1 day), 48 h (2 days), 72 h (3 days), 7 days and 14 days.

The following products were tested
sample A: a biocompatible product according to formulation 2a, wherein the divalent zinc cation 10 is added to the matrix by the incorporation of zinc gluconate at a zinc gluconate concentration of 20 ppm (which is to say, a divalent zinc cation concentration in the matrix of 0.002%).
sample B: a biocompatible product according to the invention and wherein it differs from formulations 4a and 4b only by a zinc gluconate concentration of 20 ppm (which is to say, a divalent zinc cation concentration in the matrix of 0.002%), wherein it is specified that the carboxymethyl chitosan is of plant (fungal) origin,
sample C: a biocompatible product produced according to the invention and differing from formulations 4a and 4b only by a zinc gluconate concentration of 20 ppm (which is to say, a divalent zinc cation concentration in the matrix of 0.002%), wherein it is specified that the carboxymethyl chitosan is of animal origin,
sample D: a product marketed under the brand name PERFECTHA® DERM (batch number: 190625-2) by Sinclair France SAS,
sample E: a physiological buffer solution,
sample F: a solution of sodium chloride (saline solution) at 9 g/L (which is to say, 0.9% by weight by volume).

TABLE 2
Average irritation scores according to
the ISO standard 10993-10 rating system

| | Day 1 | Day 2 | Day 3 | Day 7 | Day 14 |
|---|---|---|---|---|---|
| Sample A | 2.00 | 0.75 | 1.50 | 1.00 | 0.00 |
| Sample B | 2.00 | 0.00 | 0.00 | 0.00 | 0.00 |
| Sample C | 3.75 | 2.00 | 2.00 | 1.75 | 0.00 |
| Sample D | 8.00 | 7.25 | 7.00 | 7.00 | 3.50 |
| Sample E | 0.25 | 0.00 | 0.00 | 0.00 | 0.00 |
| Sample F | 0.00 | 0.00 | 0.00 | 0.00 | 0.00 |

Biocompatible products according to the present invention therefore have satisfactory biocompatibility in the case of an intracutaneous injection.

### Eye Irritation Testing

Ocular irritation testing was carried out (adapting ISO standard 10993—part 10) by instillation of 0.1 mL doses of test samples into the lower conjunctival sac of the left eye of rabbits, whereas a 0.1 mL dose of a 9 g/L (0.9% w/v) sodium chloride solution (saline solution) was instilled into the lower conjunctival sac of the right eye of said rabbits as a control. Observations were made in terms of ocular reactions after 1 h, 24 h (1 day), 48 h (2 days) and 72 h (3 days). Each sample was tested on two rabbits.

The following products were tested:
sample G: a biocompatible product according to formulation 2a, wherein the divalent zinc cation is added to the matrix by the incorporation of zinc gluconate at a zinc gluconate concentration of 20 ppm (which is to say, a divalent zinc cation concentration in the matrix of 0.002%).
sample H: a biocompatible product according to the invention, differing from formulations 4a and 4b only by a zinc gluconate concentration of 20 ppm (which is to say, a divalent zinc cation concentration in the matrix of 0.002%), wherein it is specified that the carboxymethyl chitosan is of plant (fungal) origin,
sample I: a biocompatible product according to the invention, differing from formulations 4a and 4b only in a zinc gluconate concentration of 20 ppm (which is to say, a divalent zinc cation concentration in the matrix of 0.002%), wherein it is specified that the carboxymethyl chitosan is of animal origin.

TABLE 3
Eye scores of sample G and of the control

| | | | 1 h | | 24 h | | 48 h | | 72 h | |
|---|---|---|---|---|---|---|---|---|---|---|
| Rabbit | Observations | | S | C | S | C | S | C | S | C |
| No. 1 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Discharge | 0 | 0 | 0 | 0 | 0 | 0 | 0 | 0 |
| No. 2 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Flow | 0 | 0 | 0 | 0 | 0 | 0 | 0 | 0 |

S: sample
C: control

TABLE 4
Eye scores of sample H and of the control

| | | | 1 h | | 24 h | | 48 h | | 72 h | |
|---|---|---|---|---|---|---|---|---|---|---|
| Rabbit | Observations | | S | C | S | C | S | C | S | C |
| No. 1 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Discharge | 0 | 0 | 0 | 0 | 0 | 0 | 0 | 0 |
| No. 2 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Discharge | 0 | 0 | 0 | 0 | 0 | 0 | 0 | 0 |

S: sample
C: control

TABLE 5
Eye scores of sample I and of the control

| | | | 1 h | | 24 h | | 48 h | | 72 h | |
|---|---|---|---|---|---|---|---|---|---|---|
| Rabbit | Observations | | S | C | S | C | S | C | S | C |
| No. 1 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Discharge | 0 | 0 | 0 | 0 | 0 | 0 | 0 | 0 |
| No. 2 | Cornea | Degree of | 0 | 0 | 0 | 0 | 0 | 0 | 0 | 0 |
| | | opacity | | | | | | | | |
| | | Area | 0 | 0 | 0 | 0 | 0 | 0 | 0 | 0 |
| | Iris | | 0 | 0 | 0 | 0 | 0 | 0 | 0 | 0 |
| | Conjunctiva | Redness | 0 | 0 | 0 | 0 | 0 | 0 | 0 | 0 |
| | | Chemosis | 0 | 0 | 0 | 0 | 0 | 0 | 0 | 0 |
| | | Discharge | 0 | 0 | 0 | 0 | 0 | 0 | 0 | 0 |

S: sample
C: control

Biocompatible products according to the present invention are non-irritating and thus have satisfactory ocular safety.

### Suitability for Use in an Eye Drop Composition

The safety of the products according to the present invention has been evaluated for use in the therapeutic treatment of dry eye syndrome, corneal injury or ocular or joint inflammation. Viscoelastic products are generally used for this purpose. Products based on hyaluronic acid, whether crosslinked or not, are widely used.

For such use, adaptive viscoelastic behavior is a major property that products must have. By a product with adaptive viscoelastic behavior, we mean a product with:
a sufficiently high viscosity at rest (which is to say, at a low shear rate) to ensure good persistence and satisfactory protection of the cornea, and
a viscosity which decreases during blinking (which is to say, at a high shear rate) to improve comfort and satisfactory distribution of the product on the ocular surface.

The evaluation was carried out through comparison in terms of viscosity with commercially available products on the market:
VisulXL® Gel of the company VISUfarma (sample J);
OPTIVE FUSION® of the company ALLERGAN (sample K);
HYLO-FORTE® of the company Ursapharm Arzneimittel GmbH (sample L).

The biocompatible products according to the present invention (after heat treatment) which were tested are:
formulation 4a (sample M),
formulation 4b (sample N).

Formulation 1 (sample O) and formulation 3 (sample P) were also tested.

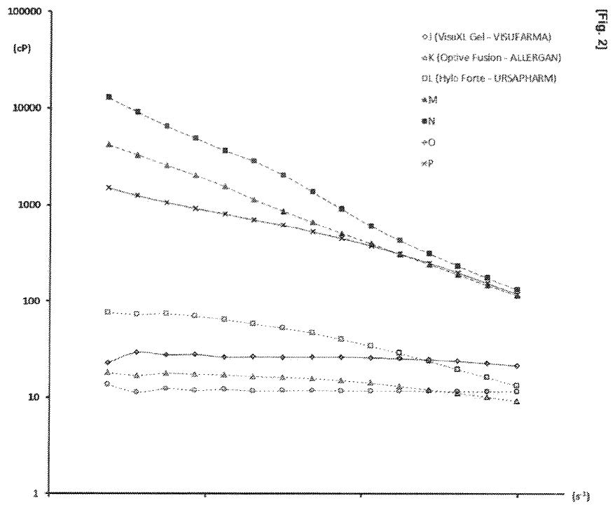

The results are shown in the form of a graph in FIG. 2.

It is firstly observed that the samples J (VisulXL® Gel of the company VISUfarma), K (OPTIVE FUSION® of the company ALLERGAN) and Q (formulation 1) show a viscoelastic behavior with very little adaptivity, with a low viscosity that is substantially constant over the entire shear rate range tested.

Whereas the sample L (HYLO-FORTE® of the company Ursapharm Arzneimittel GmbH) shows a much more adaptive viscoelastic behavior, its viscosity remains low over the entire shear rate range tested.

It can be seen that the formulations according to the present invention (samples M and N) all have a viscosity that is high at low shear rates and decreases fairly steadily (more or less rapidly) to a significantly lower viscosity at high shear rates. The formulations according to the invention thus have an adaptive viscoelastic behavior making them suitable for use in a eye drop compositions, and the concentration of divalent zinc cation in the matrix and the degree of crosslinking of the matrix make it possible to adapt the viscosity to the desired use.

The formulations according to the present invention are thus particularly interesting for therapeutic treatment by instillation on the ocular surface to prevent or combat corneal injury or dry eye syndrome, in particular with the objective of lubricating or regenerating the ocular surface.

At low shear rates, the viscosity of the formulations according to the invention (M and N) is significantly higher than the viscosity of commercially available products.

The present invention is not limited to the embodiments which have been explicitly described, but rather includes the various variants and concept descriptions thereof contained within the scope of the claims below.

1. A biocompatible product having a matrix comprising:
a polysaccharide selected from the group comprising hyaluronic acid, a salt of hyaluronic acid, chondroitin sulfate, cellulose derivatives, and a mixture thereof,
with a divalent zinc cation present in the matrix,
characterized in that:
the matrix comprises chitosan, preferably a chitosan derivative or a salt of chitosan,
in said matrix, at least part of the polysaccharide is co-crosslinked with at least a part of chitosan, of chitosan derivative, or of a salt of chitosan.

2. The biocompatible product according to claim 1, characterized in that the concentration of divalent zinc cation in the matrix is between 0.001% and 1% by weight relative to the total weight of the matrix, and preferably between 0.01% and 1% by weight relative to the total weight of the matrix.

3. The biocompatible product according to claim 1, characterized in that the concentration of divalent zinc cation in the matrix is less than or equal to 0.1% by weight relative to the total weight of the matrix.

4. The biocompatible product according to claim 1, characterized in that the concentration of divalent zinc cation in the matrix is between 0.002% and 0.06% by weight relative to the total weight of the matrix.

5. The biocompatible product according to claim 1, characterized in that the divalent zinc cation is included in the matrix by the addition of a salt of zinc or zinc saccharide, preferably zinc gluconate, zinc chloride or zinc sulfate.

6. The biocompatible product according to claim 1, characterized in that:
the concentration of polysaccharide selected from the group comprising hyaluronic acid, a salt of hyaluronic acid, chondroitin sulfate, cellulose derivatives, and a mixture thereof, is between 0.01% and 3% by weight relative to the total weight of the matrix, and/or
the concentration of chitosan, of chitosan derivative or of salt of chitosan is between 0.01% and 3% by weight, based on the total weight of the matrix.

7. The biocompatible product according to claim 1, characterized in that the chitosan derivative is a carboxyalkyl chitosan, preferably a carboxymethyl chitosan.

8. The biocompatible product according to claim 1, characterized in that the polysaccharide is hyaluronic acid or a salt of hyaluronic acid, and preferably has a molecular weight of between 0.1 MDa and 5 MDa.

9. A cosmetic or pharmaceutical composition comprising the biocompatible product according to claim 1.

10. The cosmetic or pharmaceutical composition according to claim 9, comprising a physiologically acceptable buffer, preferably containing polyols and/or phospholipids.

11. The cosmetic according to claim 9, for use in a method of cosmetic treatment or therapeutic treatment, comprising in particular topical application, implantation, instillation or injection by subcutaneous, intradermal, mucosal, ocular, intraocular or intraarticular route, of said composition.

12. The cosmetic for use according to claim 11, for the prevention and/or treatment of pathologies that may be improved or avoided by:
the filling or replacing of a biological tissue; and/or
the increase in volume of said biological tissue; and/or
the supplementation or replacement of a biological fluid.

13. The cosmetic for use according to claim 11, for a use in a method of treatment of osteoarthritis, or for the repair of a cartilage defect, wherein the composition is, for example, formulated for an administration by injection in the synovial fluid or for an implantation in cartilage after mixing with blood, or for a method of treatment of dry eye.

14. A composition according to claim 9, for use in a therapeutic, surgical or cosmetic treatment method, including in particular treatment in rheumatology, ophthalmology, gynecology, esthetic medicine, plastic surgery, internal surgery, orthopedic surgery, for the prevention of post-surgical tissue adhesions, and in dermatology.

15. The composition according to claim 9, for use in the therapeutic treatment of dry eye syndrome, of a corneal injury or ocular or joint inflammation.

16. The composition according to claim 9, for use in a therapeutic treatment during which said composition is applied by instillation to the ocular surface to prevent or combat corneal injury or dry eye syndrome, in particular for the purpose of lubricating or regenerating the ocular surface.

17. An eye drop composition comprising a biocompatible product according to claim 1.

18. A method of manufacturing a biocompatible product according to claim 1 or a composition according to claim 9, said method comprising the following steps:
a. dissolution in the same aqueous solution of:
i. a polysaccharide selected from the group comprising hyaluronic acid, a salt of hyaluronic acid, chondroitin sulfate, cellulose derivatives, and a mixture thereof, preferably hyaluronic acid or a salt of hyaluronic acid,
ii. chitosan, preferably chitosan derivative, or a salt of chitosan,
b. addition of a crosslinking agent to said aqueous solution and the provocation of a crosslinking of the mixture to form a matrix,
c. addition of a divalent zinc cation before and/or after step b.

19. The method of manufacturing according to claim 18, characterized in that the pH of the biocompatible product or composition is adjusted to between 6 and 8, preferably 7.

20. The method of manufacturing according to either claim 18 or claim 19, characterized in that the biocompatible product or composition is subjected to a sterilizing heat treatment, preferably a heat treatment at a temperature of between 120° C. and 140° C. for a time of between 1 and 20 minutes.
